(19) United States
(12) Patent Application Publication
(10) Pub. No.: US 2007/0118211 A1
(43) Pub. Date: May 24, 2007

(54) Method for preparing drug eluting medical devices and devices obtained therefrom

(76) Inventor: Gianluca Gazza, Avenue Princesse Grace 31 (MC)

Correspondence Address: HOGAN & HARTSON LLP;IP GROUP, COLUMBIA SQUARE, 555 THIRTEENTH STREET, N.W., WASHINGTON, DC, 20004, US

(21) Appl. No.: 10/577,932

(22) PCT Filed: Nov. 7, 2003
(86) PCT No.: PCT/IB03/05003

§ 371 Date: Dec. 18, 2006

(51) Int. Cl. A61F 2/02; A61K 9/50; H05H 1/24; B05D 3/00

(52) U.S. Cl. 623/1.42; 427/2.21; 424/423; 427/569

(57) ABSTRACT

The present invention relates to a method for preparing a drug eluting medical device comprising the application to a stent of a polymer having functional groups capable of chemically binding biological molecules, characterised in that said application is carried out in a single step by means of cold plasma methods. Moreover, the invention also relates to a medical device obtained therefrom.

The present invention relates to a method for preparing drug eluting medical devices and devices obtained therefrom. In particular, the invention relates to a method for preparing a vascular stent covered with one or more drugs for treating and/or preventing re-stenosis.

In angioplasty, the use of stents in treating coronary occlusions is currently well known and widely accepted and practised. Stents are reticular metal prostheses positioned in the stenotic portion of the vessel which remain at the site of the lesion after the elution system and the balloon have been withdrawn. In this way, the stent compresses the plaque and provides the vessel wall with a mechanical support in order to maintain the diameter of the vessel re-established by expanding the balloon, and prevent collapse of the vessel.

However, the long-term effectiveness of using intercoronary stents still presents the major problem of post-angioplasty coronary re-stenosis, that is the phenomenon of reocclusion of the coronary vessel. In fact, this phenomenon of re-stenosis occurs in 15-30% of patients undergoing angioplasty with stents, as described for example in Williams D O, Holubkov R, Yeh W et al. “Percutaneous coronary interventions in the current era are compared with 1985-1986: The National Heart, Lung and Blood Institute Registries”, Circulation 2000; 102:2945-2951.

Stenosis caused by insertion of the stent is due to the hyperplasia of the newly formed intima. In particular, the mechanical damage to the artery wall caused by the stent and the foreign body reaction caused by the presence of the stent produce a chronic inflammatory process in the vessel. This phenomenon gives rise in turn to the elution of cytokins and growth factors which promote activation of proliferation and migration of the smooth muscle cells (SMC). The growth of these cells together with the production of an extracellular matrix produce enlargement of the section of the vessel occupied by neointima and therefore the process of reduction in the opening of the vessel, giving rise to the above-mentioned re-stenosis.

To prevent this problem, various methods have been developed including one which provides for covering the stent directly with a drug or with a coating of the polymer type capable of incorporating the drug and eluting it locally by a controlled mechanism. A typical example of a coated stent capable of eluting drugs (DES, drug eluting stent) is described in the paper by Takeshi Suzuki and collaborators “Stent-Based Delivery of Sirolimus Reduces Neointimal Formation in a Porcine Coronary Model”, Circulation 2001; 104: 1188-1193. The materials used are generally polymers, either degradable or non-degradable which must have characteristics of adhesion to the metal substrate (stent), the ability to regulate the rate of elution of the drug, an absence of toxicity phenomena and favourable interaction with the surrounding tissue.

In particular, as far as the last characteristic is concerned, the interactions of the material with the surrounding tissue are to a large extent controlled by the surface properties of the material. The materials used in medical devices in general do not present optimum surface characteristics as far as interaction with the host tissue is concerned. This circumstance manifests itself from a clinical point of view with the onset of foreign body reaction phenomena and, in particular for materials in contact with the blood, with the formation of thrombi and/or emboli. The extent of the phenomenon is such that the thrombogenicity of synthetic materials is the most serious obstacle to the development of small-sized artificial vessels.

To attempt to overcome these disadvantages, procedures have been developed which, by means of chemical reactions, provide for the covering of the thrombogenic material with natural non-thrombogenic molecules. The anticoagulant heparin is a typical example. These procedures provide for a first step in which chemical groups suitable for binding heparin, hialuronic acid or other biomolecules are introduced onto the surface of the stent (or of the medical device in general), and a second step consisting in chemical bonding of the heparin, hyaluronic acid or other biomolecules with chemical groups introduced by means of the previous step.

Consequently, the polymers used for drug delivery are not capable as they stand of directly binding biomolecules but require the above step of introducing functional groups and subsequently immobilising said biomolecules.

There are polymers which of themselves contain functional groups such as amino groups or from which amino groups can be generated. These polymers can be applied to the surface of the stents using conventional technology.

However, it has been found that these polymers suffer from the serious disadvantage of being hydrophilic and, since the step of bonding with heparin or other biomolecules generally takes place in a solvent and in particular for heparin in an aqueous environment, there is a major risk of losing at least part of the drug during preparation of the stent precisely because of the solubility of the polymer in water; moreover, precisely because of the hydrophilic nature of the polymer, the ability to control drug elution is limited and it is entirely unsuitable for controlling elution of drugs which in their turn are hydrophilic.

Moreover, the drug eluted into the solution containing heparin and the functional groups may interfere with the immobilisation reaction, jeopardizing a successful outcome.

The problem addressed by the present invention is therefore that of making available a method of preparing a drug eluting vascular stent capable of overcoming the disadvantages mentioned above.

These problems are solved by a method for preparing a drug eluting medical device which simplifies the production procedure and at the same time avoids loss of the drug or other compounds which may jeopardize the preparation of the stent.

A first object of the invention is therefore to make available a method for preparing a medical device as outlined in the appended main claim.

A second object of the invention is that of providing a drug eluting medical device obtainable according to the above-mentioned method.

By the term “drug eluting medical device” is meant a device to be inserted in the human or animal body, internally or subcutaneously, intended to remain in said human or animal body for a defined period of time or permanently, and which is capable of eluting a pharmaceutically effective dose of one or more drugs for at least part of the time during which it resides in the human or animal body. This medical device may be a vascular device, prosthesis, probe, catheter, dental implant or similar. More preferably, this device will be a vascular stent.

Other characteristics and advantages of the present invention will become clear from the following description of an embodiment provided by way of non-limiting example, in which:

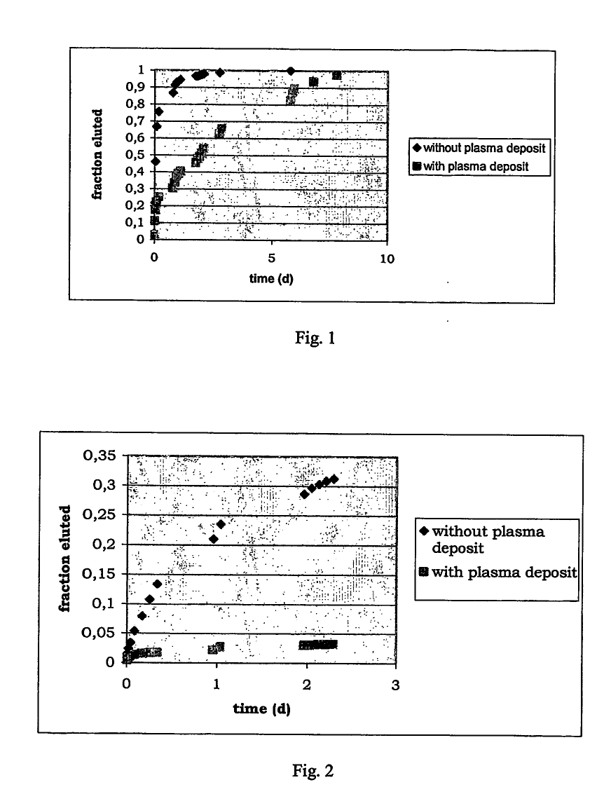

FIG. 1 shows the elution curve for a hydrophilic drug from a stent covered with polymer according to the state of the art compared with the elution curve for a hydrophilic drug from a stent covered with polymer according to the invention;

FIG. 2 shows the elution curve for a hydrophobic drug from a stent covered with polymer according to the state of the art compared with the elution curve for a hydrophobic drug from a stent covered with polymer according to the invention.

Following numerous experiments, it was surprisingly found that if polymers having functional groups such as amino groups were applied to the surface of the medical device in a single step using a cold plasma method, coverage of the stent was obtained in the form of a hydrophobic film, adhering well and with active and stable functional groups capable of rapid binding of heparin, hialuronic acid or other biomolecule.

The following description will relate to a vascular stent, but could also be applied to any other medical device of the invention.

In particular, it has been observed that polymers with amino functional groups deposited on the metal surface of vascular stents by cold plasma assume characteristics of hydrophobicity, excellent adhesion to the stent, a high degree of cross-linking so as to operate as a barrier slowing the diffusion of a drug and the ability to bind heparin and other biomolecules by means of said amino groups.

The method for preparing a drug eluting vascular stent as disclosed in the invention therefore comprises application to the surface of said stent of a polymer having stable reactive functional groups, such as for example amino, carboxyl and sulphhydryl groups, in which this application takes place in a single step by means of cold plasma methods.

According to a first form of embodiment, the polymers are deposited in the form of a film. In particular, said polymers have functional groups capable of forming a covalent bond with said biological molecules, preferably chosen from among heparin, hyaluronic acid or anti-thrombotic substances in general. More particularly, said polymers are chosen from the group constituted by polymers containing amino, carboxyl and sulphhydryl groups. Preferably, the polymers with amino groups are derived from precursors or monomers chosen from among allylamine, heptylamine, aliphatic or aromatic amines; polymers with carboxyl groups are derived from precursors or monomers chosen from between acrylic acid and methacrylic acid. Polymers with sulphhydriyl groups are derived from precursors or monomers chosen from among volatile mercaptans.

The method disclosed by the invention may also provide for further polymer layers to be deposited depending on the degree or type of mechanisms for elution of the drug which it is wished to obtain. These latter deposits are produced according to methods known in the art such as immersion in a suitable solution or spraying with a pneumatic spray gun or using the above-mentioned cold plasma method. It should be noted that in any case the outermost layer must be deposited according to the cold plasma method using the above-mentioned polymers having functional groups.

The plasma used according to the invention is a cold plasma, that is the temperature of the total mass of gas in the plasma phase is of the same order as the ambient temperature. Said plasma is generated in a conventional reactor of the type comprising a treatment chamber inside which there is a support for the material to be treated, with a discharge source located nearby to produce the plasma.

The cold plasma may be produced under vacuum or at atmospheric pressure and may be generated using various electromagnetic sources, that is sources of various frequencies and various geometries, such as for example radiofrequency generators or microwave generators, with electrodes of the inductive or capacitive type.

In general, when the vacuum method is used, the cold plasma is produced in a chamber with a pressure which may vary between 0.01 and 10 mbar.

As far as the conditions of treatment are concerned, these depend on the electrical power which may vary from 1 to 500 W, on the geometry of the source which produces the plasma which may be inductive or capacitive and on the frequencies of the electromagnetic radiation used to produce the plasma which may be in the microwave or radiofrequency range.

Moreover, the cold plasma which is generated is characterised by a charged species density of between 10⁸ and 10¹² cm⁻³, a condition of substantial neutrality of charges (quasi-neutral, ion density≈electron density), electron energies from 0.1 to 10 eV or mean electrical energy calculated as (ekBT/m)1/2 (e=1.9 10-19 C, kB=1.38 10-23 J/K, m=9.1 10-31 kg, T=absolute temperature in Kelvin), while the ions and the neutral particles are at temperatures of the order of ambient temperature.

The treatment time in a cold plasma is generally not more than 30 minutes, is preferably between 0.1 and 20 minutes and still more preferably between 1 and 10 minutes.

Preferably, the plasma treatment under vacuum takes place according to a discontinuous or continuous method. Said method will not be described in detail here since it is widely known in the art.

The cold plasma used may preferably be generated at a pressure of less than atmospheric pressure. The precursor or monomer which will be polymerized in the plasma phase is introduced into the reactor in the form of gas or vapour, with flow rates which vary from 0.1 to 200 sccm (cubic centimetres in standard conditions per minute). At this point, the plasma is initiated and the treatment is carried out.

A preferably conventional type of reactor, not shown, according to the invention is represented by a radiofrequency plasma reactor, with parallel flat plate electrodes, comprising a treatment chamber of steel, aluminium or glass, connected to a vacuum pump. The precursor or monomer is introduced in the form of gas or vapour inside the chamber by means of a suitable feed system, and a potential difference is applied between the electrodes. In this way, the flow of gas or vapour is ionized, triggering the series of reactions which leads to its being deposited according to the methods typical of plasma polymerisation. The precursor or monomer which gave the best results was allylamine since the presence of the double bond substantially increases the speed of deposition and therefore the speed with which the optimum thicknesses for use are reached. In particular, the thicknesses which are generally used for a drug eluting polymer are in fact between 0.01 micron and 10 microns. Preferably, as far as allylamine is concerned, the thicknesses vary from 0. 1 to 10 microns.

According to a variant embodiment of the invention, the method for preparing a vascular stent also comprises, before the polymer comprising functional groups is deposited by cold plasma, a step of applying at least one layer of drug incorporated where appropriate in a polymer capable of eluting said drug. This step is carried out using conventional methods such as immersion or spraying and using conventional polymers.

The nature of the polymers normally used for this step is substantially dictated by the elution mechanism envisaged for the drug and, in any case, within the scope of a person skilled in the art. For example, in the case of coronary stents for which elution times of the order of months are required, it will be essential to use polymers which produce a slow elution mechanism. In the case of hydrophilic drugs, such as imatinib mesilate (sold under the name of Glivec® by the Novartis company), it will be preferable to use hydrophobic hydrocarbon polymers such as polystyrene, polyethylene, polybutadiene and polyisoprene. Polybutadiene, because of its elastomeric nature, the absence of toxic effects and its availability is the preferred polymer. In the case of hydrophobic drugs, such as taxol, tacrolimus and similar or dexamethasone, more hydrophilic polymers may be used, such as hydrophilic polyamides, polyurethanes, polyacrylates or polymethacrylates. Polyhydroxy-butylmethacrylate and polyhydroxyethylmethacrylate applied alone or with the hydrophobic component polybutadiene, so as to regulate the elution mechanism more finely, are the preferred polymers.

As described previously, these polymers will preferably be applied in the form of a solution in organic solvents by immersion or spraying. In particular, the technique of spraying by means of an airbrush or similar air-operated systems, or the technique of spraying using ultrasound nozzles may be used.

The thickness of the layer deposited depends on the nature of the drug, the polymer and the elution mechanism desired. In any case, indicative values for a person skilled in the art are between 0.5 and 20 microns, preferably between 1 and 10 microns. Adjustments on the basis of what has been stated are in any case part of the state of the art.

As far as the drug to be eluted is concerned, in general all drugs known for the purpose may be used. In particular, anti-inflammatory, anti-proliferative, anti-migratory drugs or immunosuppressive agents may be used. Preferably, imatinib mesilate may be used, that is 4-[(4-methyl-1-piperazinyl)methyl]-N-[4-methyl-3-[[4- (3-pyridinyl)-2-pyrimidinyl]amino]-phenyl]benzamide methanesulphonate, marketed under the name Glivec® by the Novartis company.

The quantity of drug to be combined with the polymer varies according to the class of drug. For example, when the drug is an anti-inflammatory, it is usually present in quantities of between 0.001 mg and 10 mg per device. When the drug is an anti-proliferative, it is present in quantities of between 0.0001 and 10 mg per device. When the drug has an anti-migratory action it may be present in quantities of 0.0001 mg to 10 mg per device. When the drug is an immunosuppressant, it is present in quantities of 0.0001 mg to 10 mg by weight per device. When the drug is imatinib mesilate (Glivec®) it is present in quantities of 0.001 mg to 10 mg per device.

The method for preparing a medical device according to the invention also comprises a step of binding/immobilising anti-thrombotic substances on the surface of the polymer bearing the functional groups. In particular, this deposit consists in chemically bonding the heparin or hyaluronic acid, for example, to amino groups of the polymer which is deposited in turn on the stent using the cold plasma technique.

Preferably, the anti-thrombotic substance is deposited by immersing the stent covered with polymer by the cold plasma method with functional groups in an aqueous solution for example of heparin or hyaluronic acid. The aqueous solution generally used comprises from 0.01% to 1% by weight of heparin or hyaluronic acid. This solution is generally prepared by dissolving 0.01 g to 1 g of heparin, for example, in 100 cc of a buffer, such as a phosphate buffer, for example, and adding 0.001 g to 1 g of a substance with an oxidizing action, such as sodium periodate. After a period of time of between 6 and 20 hours remaining in solution, from 20 to 200 cc of a buffer solution such as a 0.001-0.1% acetic acid-sodium acetate solution are added. From 1 to 10 cc are then taken from said solution and placed in a suitable receptacle such as a Petri dish. The stent is then immersed in the dish and 0.001 to 0.01 g of a substance with a reducing action, such as sodium cyanoborohydride, is added. After a period of time of not more than 30 minutes, preferably between 15 and 30 minutes, the stent is removed and washed with water. It is then dried in an oven.

According to a further variant embodiment of the invention, further biodegradable layers may be applied, with or without a drug, over the layer of heparin, hyaluronic acid or other immobilised molecules which as a result of their normal process of degradation expose the heparin, hyaluronic acid or said other immobilised biomolecules.

The method according to the invention may also comprise a preliminary step of cleaning and/or washing the surface of the stent so as to prepare it for the above-mentioned steps of deposition. Generally, the cleaning/washing step consists in treating with degreasing solutions, such as organic solvents or water/isopropyl alcohol mixtures, or treating with cold plasma of air or argon.

This preliminary step may in addition be followed by at least one pretreatment step to promote adhesion of the drug, where appropriate bound to an elution polymer, or of subsequent layers. In general, the pretreatment step may include treatment with cold plasma of air or oxygen, or the deposition by plasma of organic layers which function as adhesion promoters between the stent and the material to be deposited.

From what has been described so far, it is clear that the method for preparing a medical device according to the present invention eliminates the step of treatment of the drug eluting polymer required to insert on its surface functional groups that are such as to allow bonding with biomolecules. In fact, this step is eliminated because of the deposition of a particular class of polymers selected precisely for their characteristics of already possessing such groups when deposited using cold plasma technology. Moreover, combining it with the use of the cold plasma method advantageously enables the polymer to be deposited without damaging the characteristics of its functional groups.

In addition to the above-mentioned examples of the method for preparing the medical device, the polymers selected and deposited by cold plasma promote bonding with biomolecules such as heparin and ensure that they are held in situ, preventing dispersion in the aqueous environment during preparation of the device.

It has also been observed that with cold plasma deposition of the polymers having functional groups as described above, the relevant drug is eluted more slowly, thus producing a barrier effect. Consequently, this effect permits a more lasting anti-stenotic action on the part of the drug.

A second object of the present invention is to make available a drug eluting medical device obtainable according to the method described previously.

In particular, said medical device may for example comprise a device structure, at least one first layer covering the surface of said structure comprising a drug, at least one second layer covering said at least one first layer comprising a polymer having stable reactive functional groups and a biological molecule layer applied to said at least one second layer by means of bonding with said functional groups, in which said at least one second layer of polymer having functional groups is deposited on said at least one first layer of drug by means of the cold plasma method.

Preferably, said at least one first layer of drug comprises a drug eluting polymer as described previously. The drug may be chosen from among the drugs listed with reference to the method for preparing the stent.

Said at least one second layer of polymer having functional groups may be selected from among the polymers mentioned previously and may be deposited according to the cold plasma method referred to above.

Also, as regards the biomolecule applied to the outer surface of the stent, this may preferably be represented by though not limited to any one of the substances described previously.

The use of polymers having functional groups for covering vascular stents by means of cold plasma methods is also an object of the present invention. Preferably, said polymers are the polymers specified previously.

From what has been stated so far, the medical devices prepared according to the above-mentioned method are seen to be particularly advantageous compared with the devices criticised in the introductory part of the present description, particularly where the drug elution mechanism is concerned. In fact, it has been observed that the stents disclosed in the invention allow more controlled elution of the drug because of the particular layer of polymer with functional groups which in some way acts as a far more active barrier compared with the polymers of the state of the art.

In addition, the polymers deposited by plasma have excellent adhesion to the vascular stent and at the same time have proved completely free of toxic phenomena.

Below, some embodiments of the invention are described purely by way of non-limiting example.

### EXAMPLE 1

### Comparison Between the Elution Mechanism of a Hydrophilic Drug from a Stent Covered with a Polymer According to the State of the Art and the Mechanism from a Stent Covered with Polymer According to the Invention

From capsules of the drug Glivec® 10 mg of the active principle imatinib mesilate were extracted by dissolving in water, filtering to remove the insoluble excipients using Albet 400 filter paper (43-38 micron) and evaporating the water using a Rotavapor (Heidolph) so as to recover the active principle in powder form. Two stainless steel stents 11 mm in length produced by the INVATEC company were coated using an Artis I airbrush (Efbe, Germany) in the following manner.

Firstly, 1 cc of a 0.250% solution in cyclohexane of polybutadiene sold by the Aldrich company having a mean molecular weight of 420,000 was applied. Following this, 1 cc of a solution obtained by dissolving 10 mg of Imatinib Mesilate (IM) in 1 cc of methanol was applied. Then 1 cc of a 0.5% solution of polybutadiene in cyclohexane, as specified above, was applied. Finally, 1 cc of a 0.5% solution in cyclohexane of polybutadiene with a molecular weight of between 1,000,000 and 4,000,000 was applied.

At this point, one of the two stents was placed in a EUROPLASMA reactor and underwent a cycle of plasma deposition of allylamine (introduced as vapour from an external receptacle which contained it as a liquid) for 8 minutes with the reactor switched to a power of 200 W at a pressure of 0.2 mbar.

Next, the stents were immersed in test tubes containing 1 cc of physiological solution and the rate of elution of the drug was measured by acquiring the visible UV spectrum using a Unicam 8700 spectrophotometer and reading off the absorbance at 261 nm. The correlation between absorbance and concentration was established by measuring the absorbance of solutions of known concentration (calibration curve). The drug elution measurements were carried out at fixed time intervals and the physiological solution was changed at each measurement. The elution curves shown in FIG. 1 were obtained.

In particular, FIG. 1 shows that deposition of the polymer by cold plasma significantly delays the elution of the hydrophilic drug compared with the elution deriving from application of a polymer according to the state of the art.

### EXAMPLE 2

### Comparison Between the Elution Mechanism of a Hydrophobic Drug from a Stent Covered with a Polymer According to the State of the Art and the Mechanism from a Stent Covered with Polymer According to the Invention

The same procedure described in Example 1 was repeated here with the difference that a hydrophobic drug, dexamethasone, was used.

10 mg of dexamethasone were dissolved in 1 cc of ethanol and applied as described previously. The elution curves were again measured as described in example 1 and the absorbance at 264.4 nm was read off. The results shown in FIG. 2 were obtained.

It should be noted that in this case, too, the polymer of allylamine deposited by cold plasma provides a notable reduction in the mechanism of elution of the drug.

### EXAMPLE 3

### Comparison of the Degree of Hydrophilicity Between a Metal Stent Treated with Heparin and a Metal Substrate without Heparin

A stent prepared according to example 1 with allylamine deposited by cold plasma underwent a process of bonding with heparin in the following manner.

0.5 g of heparin (Bioiberica) was dissolved in 100 cc of phosphate buffer and 0.016 g of sodium periodate (Sigma-Aldrich) was added. After 16 hours of remaining in solution, 100 cc of 0.05% acetic acid-sodium acetate solution were added. 5 cc of this solution were taken and placed in a Petri dish. The stent was then immersed in the dish and 0.01 g of sodium cyanoborohydride (Sigma-Aldrich) were added. After 30 minutes, the stent was removed and washed with water. It was then dried in an oven. At this point, the stent was far more hydrophilic compared with a non-heparinized stent precisely because of the presence of heparin bonded onto its surface.

To provide an analytical base, the same treatment as just described was carried out on plates of ASI 316 L steel of side 1 cm, that is the material of which the stent was constituted. A heparinized plate was compared with a non-heparinized plate by a comparison using X-ray Photoelectron Spectroscopy (XPS) analysis to supply the chemical composition of the surface layer. The XPS analysis was carried using a Perkin Elmer PHI 5500 ESCA System instrument. The result of the analysis expressed in atomic % is given in table 1 below.

TABLE 1

| | | | | | | Other |
|---|---|---|---|---|---|---|
| Specimen | C | O | N | S | Si | (<1%) |
| Non- | 78.4 | 10.7 | 9.4 | — | 1.3 | Na, P |
| heparinized | | | | | | |
| plate | | | | | | |
| Heparinized | 69.2 | 21.9 | 2.4 | 3.2 | 1.9 | Mg, Cl, Na |
| plate | | | | | | |

Compared with the untreated specimen, the specimen treated with heparin shows an increase in the O/C ratio and in S concentration expected in the heparinization processes.

### EXAMPLE 4

### Comparison of the Degree of Hydrophilicity Between a Metal Stent Treated with Hyaluronic Acid and a Metal Stent without Hyaluronic Acid

A stent prepared according to example 1 with allylamine deposited by cold plasma underwent a process of bonding with hyaluronic acid in the following manner.

0.5 g of hyaluronic acid (Lifecore) was dissolved in 100 cc of deionized water. 5 cc of said solution were taken and placed in a Petri dish. The stent was then immersed in the dish and 0.03 g of N-hydroxy succinimide and 0.04 of dimethyl carbodiimide (EDC) (both Sigma-Aldrich) were added. After 30 minutes, the stent was removed and washed with water. It was then dried in an oven. At this point, the stent was far more hydrophilic compared with a stent not covered with hyaluronic acid precisely because of the presence of hyaluronic acid bound onto its surface.

### EXAMPLE 5

### Production of a Stent Covered with Polymer According to the Invention, with Immobilisation of Hyaluronic Acid and Further Covering with a Biodegradable Hyaluronic Acid Derivative-Based Layer

From capsules of the drug Glivec® 10 mg of active principle imatinib mesilate were extracted by dissolving in water, filtering to remove the insoluble excipients and evaporating the water as described in example 1. Two stainless steel stents 11 mm in length produced by the INVATEC company were coated using an Artis I airbrush (Efbe, Germany) in the following manner.

Firstly, 1 cc of a 0.250% solution in cyclohexane of polybutadiene (Aldrich, mean molecular weight 420,000) was applied. Following this, 1 cc of solution obtained by dissolving 10 mg of Imatinib Mesilate (IM) in 1 cc of methanol was applied. Then 1 cc of 0.5% solution of polybutadiene (details as previously) in cyclohexane was applied. Finally, 1 cc of a 0.5% solution in cyclohexane of polybutadiene with a molecular weight of between 1,000,000 and 4,000,000 was applied.

At this point, one of the two stents was placed in a EUROPLASMA reactor for the plasma treatment and underwent a cycle of plasma deposition of allylamine (introduced as vapour from an external receptacle which contained it as a liquid) for 8 minutes with the reactor switched to a power of 200 W at a pressure of 0.2 mbar.

Next, 0.5 g of hyaluronic acid (Lifecore) was dissolved in 100 cc of deionized water. 5 cc of said solution were taken and placed in a Petri dish. The stent was then immersed in the dish and 0.03 g of N-hydroxy succinimide and 0.04 of dimethyl carbodiimide (EDC) (both Sigma-Aldrich) were added. After 30 minutes, the stent was removed and washed with water and dried. At this point, a layer was applied of a hyaluronic acid derivative insoluble in water and degradable, the total benzyl ester HYAFF 11) (Fidia Advanced Biopolymers, Abano Terme, Italy). This material, together with the drug imatinib mesilate, was applied from a solution of 0.2% HYAFF and 1% IM in hexafluoroisopropanol using an airbrush.

In this way, a stent is obtained which elutes the drug from the surface layer of HYAFF and from the underlying layer, in which the surface layer will degrade in situ leaving exposed the surface on which the hyaluronic acid is bonded to the barrier and functional layer deposited by plasma.

1. A method for preparing a drug eluting medical device comprising the application to said device of a polymer having active functional groups capable of chemically binding biological molecules, characterised in that said application takes place in a single step by means of cold plasma methods.

2. A method according to claim 1, in which said polymers are chosen from among polymers having amine groups, carboxyl groups and sulphhydryl groups.

3. A method according to claim 2 in which the precursors of said polymers having amine groups are chosen from among allylamine, heptylamine, aliphatic amines and aromatic amines.

4. A method according to claim 2 in which the precursors of said polymers having carboxylic groups are chosen from between acrylic acid and methacrylic acid.

5. A method according to claim 2, in which the precursors of said polymers having sulphhydryl groups are chosen from among volatile mercaptans.

6. A method according to claim 1, in which said cold plasma methods comprise cold plasma produced under vacuum using discontinuous or continuous technology.

7. A method according to claim 6, in which said cold plasma under vacuum is generated at a pressure which may vary between 0.01 and 10 mbar, at a power of between 1 and 500 W and for a period of time of not more than 30 minutes.

8. A method according to claim 1, in which said cold plasma methods consist in cold plasma produced at atmospheric pressure.

9. A method according to claim 1 in which the precursor of said polymer is in the form of a gas.

10. A method according to claim 1, in which the precursor of said polymer is in the form of a vapour.

11. A method according to claim 1, in which said polymer is applied in the form of film with a thickness of between 0.01 and 10 microns.

12. A method according to claim 1, also comprising before the application of said polymer having functional groups a step of applying at least one layer of a drug incorporated where appropriate in a polymer capable of eluting said drug.

13. A method according to claim 12, in which said drug is chosen from the group consisting of anti-inflammatory, anti-proliferative and anti-migratory drugs and immunosuppressive agents.

14. A method according to claim 13, in which said drug is 4-[(4-methyl-1-piperazinyl)methyl]-N-[4-methyl-3-[[4-(3-pyridinyl)-2-pyrimidinyl]amino]-phenyl]benzamide methanesulphonate.

15. A method according to claim 12, in which the drug eluting polymer is chosen from among hydrophobic hydrocarbons, polyamides, polyacrylates and polymethacrylates.

16. A method according to claim 15 in which said hydrophobic hydrocarbons are chosen from among polystyrene, polyethylene, polybutadiene and polyisoprene.

17. A method according to claim 15, in which said polymer is chosen from among polyhydroxybutylmethacrylate, polyhydroxyethylmethacrylate, where appropriate in combination with polybutadiene.

18. A method according to claim 12 in which said drug which may be incorporated in a drug eluting polymer is applied by means of immersion in a suitable solution or deposited by spraying.

19. A method according to claim 18 in which said drug eluting polymer is deposited in the form of film with a thickness of between 0.5 and 20 microns.

20. A method according to claim 12, in which when said drug is an anti-inflammatory, it is present in quantities of between 0.001 mg and 10 mg per device.

21. A method according to claim 12, in which when said drug is an anti-proliferative, it is present in quantities of between 0.0001 and 10 mg per device.

22. A method according to claim 12, in which when said drug has an anti-migratory action, it is present in quantities of between 0.0001 mg and 10 mg per device.

23. A method according to claim 12, in which when the drug is an immunosuppressant, it is present in quantities of between 0.0001 mg and 10 mg per device.

24. A method according to claims 1 in which when said drug is 4-[(4-methyl-1-piperazinyl)methyl]-N-[4-methyl-3-[[4-(3-pyridinyl)-2-pyrimidinyl]amino]-phenyl]benzamide methanesulphonate, it is present in quantities of between 0.001 mg and 10 mg per device.

25. A method according to claim 1, also comprising a step of depositing biological molecules on the surface of said polymer having stable reactive functional groups.

26. A method according to claim 25, in which said biological molecules are chosen from among anti-thrombotic substances and hyaluronic acid.

27. A method according to claim 26, in which said biological molecules are heparin.

28. A method according to claim 26, in which said biological molecules are deposited by immersing the medical device in an aqueous solution containing said biological molecules in a concentration of 0.01% to 1% by weight.

29. A method according to claim 1, also comprising a preliminary step of cleaning/washing said medical device.

30. A method according to claim 29, in which said preliminary cleaning/washing step is followed by a step of pretreatment of said medical device to promote adhesion of the drug incorporated where appropriate in an eluting polymer to this device.

31. A method according to claim 1, also comprising the application of further biodegradable polymer layers over said biological molecule layer.

32. A method according to claim 1, comprising in succession the application of at least one first layer of 4-[(4-methyl-1-piperazinyl)methyl]-N-[4-methyl-3-[[4-(3-pyridinyl)-2-pyrimidinyl]amino]-phenyl]benzamide methanesulphonate included where appropriate in a polymer to the surface of said medical device, the application by cold plasma of at least one second layer of polymer of allylamine, the bonding of heparin to said at least one second layer and application of at least one third layer of biodegradable polymer onto said heparin.

33. A drug eluting medical device obtainable by the step of applying to said device a polymer having active functional groups capable of chemically binding biological molecules, characterised in that said application takes place in a single step by means of cold plasma methods.

34. The medical device according to claim 33, comprising a device structure, at least one first layer covering the surface of said structure comprising a drug, at least one second layer covering said at least one first layer comprising a polymer having stable reactive functional groups and a biological molecule layer bonded to said at least one second layer by means of chemical bonding with said functional groups, in which said at least one second layer of polymer is deposited on said at least one first layer by means of a cold plasma method.

35. A The medical device according to claim 34, in which said drug is a drug chosen from the group consisting of anti-inflammatory, anti-proliferative and anti-migratory drugs and immunosuppressive agents.

36. The medical device according to claim 34, in which said drug eluting polymer is one or more of polystyrene, polyethylene, polybutadine and polyisopsene.

37. The medical device according to claim 34, in which said polymer having stable reactive functional groups is one of the polymers described in claim 2.

38. The medical device according to claim 34, in which said biological molecule is chosen from among anti-thrombotic substances and hyaluranic acid.

39. A medical device according to claim 34, said device being chosen from among vascular devices, prostheses, probes, catheters, dental implants or similar.

40. A medical device according to claim 39, said device being a vascular stent.

41. The use of polymers having reactive functional groups chosen from among the polymers described in claim 2, for covering medical devices, preferably vascular stents, by means of cold plasma methods of deposition.
